(19) United States
(12) Patent Application Publication
(10) Pub. No.: US 2012/0085350 A1
(43) Pub. Date: Apr. 12, 2012

(54) Inner Type Tracheostomy Tube

(76) Inventors: Ti-Li Chang, Hou-Li Hsiang (TW); Sheng-Yu Chiu, Hou Li Hsiang (TW)

(21) Appl. No.: 12/899,624

(22) Filed: Oct. 7, 2010

(51) Int. Cl. A61M 16/04

(52) U.S. Cl. 128/207.14

(57) ABSTRACT

An inner type tracheostomy tube has an outer tube and an inner cannula. The outer tube has an airway tube, an inflatable cuff, an inflating tube, a neck mount and a holding mount. The neck mount is mounted around the airway tube. The holding mount is formed on the neck mount around the airway tube and has a holding ring and two holding arms. The holding ring is formed on the neck mount. The holding arms are formed on the holding ring and protrude from the holding ring and each has a holding recess. The inner cannula is connected to the outer tube and has an inner tube, a disk segment and two locking tabs. The disk segment is formed on the inner tube. The locking tabs are formed on the disk segment, are respectively connected to the holding arms and each has a locking portion and a pressing button.

### BACKGROUND OF THE INVENTION

1. Field of the Invention

The present invention relates to an inner type tracheostomy tube, and more particularly to an inner type tracheostomy tube that can easily and safely change an inner cannual of the inner type tracheostomy tube.

2. Description of Related Art

A patient who has dyspnea due to trachea obstruction or disease caused by weakness or drug effects may require a conventional tracheostomy tube inserted into the trachea through a stoma formed through the trachea to provide air from a respiratory tube directly to the patient through the conventional tracheostomy tube.

When changing the conventional tracheostomy tube, a surgical operation is needed to inconveniently operate on the patient under a general anesthesia. A conventional inner type tracheostomy tube can be used to improve the aforementioned problem and has an outer tube and an inner cannula. The inner cannula is connected to the outer tube by screwing or clamping. Thus, the inner cannula of the conventional inner type tracheostomy tube can be changed from the outer tube to improve of the convenience of using the conventional tracheostomy tube.

However, the inner cannula of the conventional inner type tracheostomy tube is screwed with the outer tube and may be separated from the outer tube when the inner cannula is rotated with a respiratory tube and this is dangerous in use. Furthermore, when the inner cannula of the conventional inner type tracheostomy tube is connected to the outer tube by an exerted clamping structure, dirt may be accumulated on the exerted clamping structure causing infection. In addition, the patent may knock the exerted clamping structure of the inner cannula to cause the inner cannula to separate from the outer tube and this is unsafe in use.

To overcome the shortcomings, the present invention tends to provide an inner type tracheostomy tube to mitigate or obviate the aforementioned problems.

### SUMMARY OF THE INVENTION

The main objective of the present invention is to provide an inner type tracheostomy tube that can easily and safely change an inner cannual of the inner type tracheostomy tube.

The inner type tracheostomy tube in accordance with the present invention has an outer tube and an inner cannula. The outer tube has an airway tube, an inflatable cuff, an inflating tube, a neck mount and a holding mount. The inflatable cuff is mounted around an inner end of the airway tube. The inflating tube is connected to and communicates with the inflatable cuff. The neck mount is securely mounted around an outer end of the airway tube. The holding mount is formed on the neck mount around the outer end of the airway tube and has a holding ring and two holding arms. The holding ring is formed on the neck mount. The holding arms are formed oppositely on the holding ring and protrude from the holding ring and each holding arm has a holding recess. The inner cannula is detachably connected to the outer tube and has an inner tube, a disk segment, two locking tabs, a connecting mount and a connector. The disk segment is formed on the inner tube and abuts the holding ring of the holding mount. The locking tabs are formed on the disk segment, are respectively connected to the holding arms and each locking tab has a shape corresponding to the holding recess, a locking portion and a pressing button. The connecting mount is securely mounted around the disk segment. The connector is securely mounted around the connecting mount.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an inner type tracheostomy tube in accordance with the present invention;

FIG. 2 is an exploded perspective view of the inner type tracheostomy tube in FIG. 1;

FIG. 3 is an enlarged exploded perspective view of the inner type tracheostomy tube in FIG. 1;

FIG. 4 is an enlarged cross sectional side view of the inner type tracheostomy tube in FIG. 1;

FIG. 5 is a side view of the inner type tracheostomy tube in FIG. 1; and

FIG. 6 is an optional side view of an inner cannula of the inner type tracheostomy tube in FIG. 1.

### DETAILED DESCRIPTION OF PREFERRED EMBODIMENT

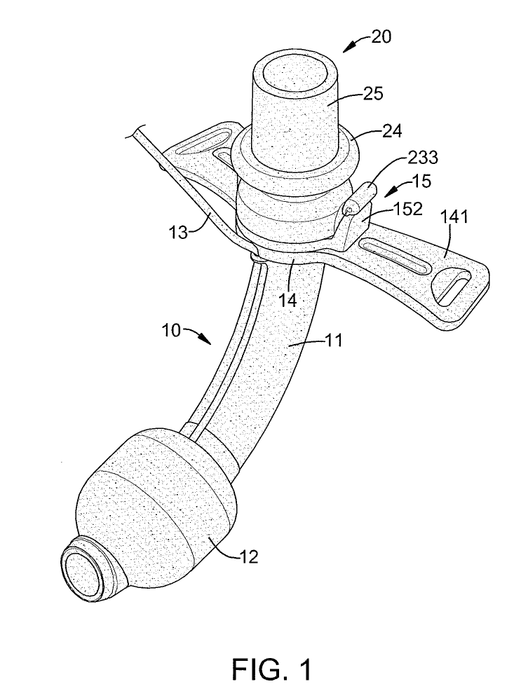

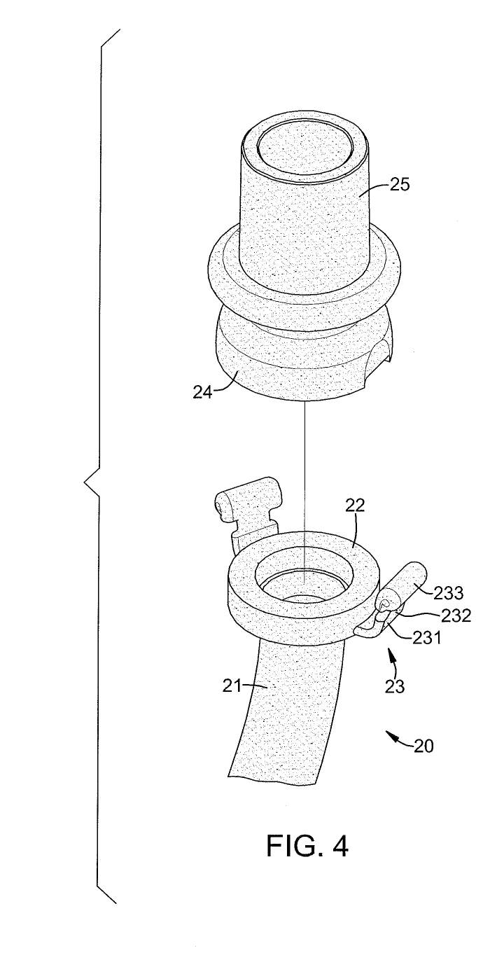

With reference to FIGS. 1 to 5, an inner type tracheostomy tube in accordance with the present invention has an outer tube 10 and an inner cannula 20.

The outer tube 10 has an airway tube 11, an inflatable cuff 12, an inflating tube 13, a neck mount 14 and a holding mount 15.

The airway tube 11 may be a hard tube and has an inner end, an outer end and an external surface. The inflatable cuff 12 is mounted around the external surface near the inner end of the airway tube 11 and is inflated to form a seal in a patient's neck for mechanical breathing and need to be totally deflated when insert into or withdraw from patient's trachea. The inflating tube 13 is connected to and communicates with the inflatable cuff 12 to inflate or deflate the inflatable cuff 12.

The neck mount 14 may be an annular hard board, is securely mounted around the external surface of the airway tube 11 near the outer end of the airway tube 11 and has a top, two sides and two neck plates 141. The neck plates 141 are elongated and are respectively formed on and protrude from the sides of the neck mount 14 to allow a cord or strap to be connected to the neck plates 141 and are mounted around the patient's neck to hold the neck plates 141 in place and hold the outer tube 10 securely in the neck of the patient.

The holding mount 15 is formed on the neck mount 14 around the outer end of the airway tube 11 and has a holding ring 151 and two holding arms 152. The holding ring 151 is formed on the top of the neck mount 14 around the outer end of the airway tube 11 and has an external surface and a top. The holding arms 152 are formed oppositely on the external surface of the holding ring 151 and protrude from the top of the holding ring 151, and each holding arm 152 has a bottom, a top, an inner face, a holding recess 153 and an opening 154. The bottoms of the holding arms 152 are formed on the external surface of the holding ring 151 and respectively abut the neck plates 141 of the neck mount 14. The tops of the holding arms 152 extend above the top of the holding ring 151.

The holding recesses 153 may be rectangular and are respectively formed in the inner faces of the holding arms 152 between the tops of the holding ring 151 and the holding arms 152, and each holding recess 153 has a width. The openings 154 are respectively formed through the tops of the holding arms 152 and respectively communicate with the holding recesses 153 of the holding arms 152, and each opening 154 has a width smaller than the width of the holding recess 153.

The inner cannula 20 is detachably connected to the outer tube 10 and has an inner tube 21, a disk segment 22, two locking tabs 23, a connecting mount 24 and a connector 25.

The inner tube 21 is mounted in the airway tube 11 of the outer tube 10 and has an inner end, an outer end and an external surface. The inner end of the inner tube 21 is mounted in the airway tube 11 near the inner end of the airway tube 11. The outer end of the inner tube 21 extends out of the holding ring 151 of the holding mount 15. The disk segment 22 is formed on the external surface of the inner tube 21 at the outer end of the inner tube 21, abuts the holding ring 151 of the holding mount 15 and has a bottom, a top and an external surface. The bottom of the disk segment 22 abuts the top of the holding ring 151.

The locking tabs 23 are formed on the external surface of the disk segment 22 from the bottom of the disk segment 22 and protrude from the top of the disk segment 22 and are respectively connected to the holding arms 152 of the holding mount 15. Each locking tab 23 has a shape corresponding to the holding recess 153, a lower end, an upper end, a middle, a locking portion 231, a neck portion 232 and a pressing button 233. The locking portions 231 are respectively formed on the lower ends of the locking tabs 23, are formed on the external surface of the disk segment 22 and are respectively and securely mounted in the holding recesses 153 of the holding arms 152, and each locking portion 231 has a width. The neck portions 232 are respectively formed on the middles of the locking tabs 23, are respectively connected with the locking portions 231 and extend out of the openings 154 of the holding arms 152. Each neck portion 232 has a width smaller than the width of the locking portion 231. The pressing buttons 233 are respectively formed on the upper ends of the locking tabs 23 and are respectively connected with the neck portions 232 above the openings 154 of the holding arms 152.

The connecting mount 24 may be soft, is securely mounted around the disk segment 22 between the locking tabs 23 and has a bottom end and a top end. The bottom end of the connecting mount 24 is securely mounted around the disk segment 22 between the locking tabs 23. The connector 25 is securely mounted around the top end of the connecting mount 24.

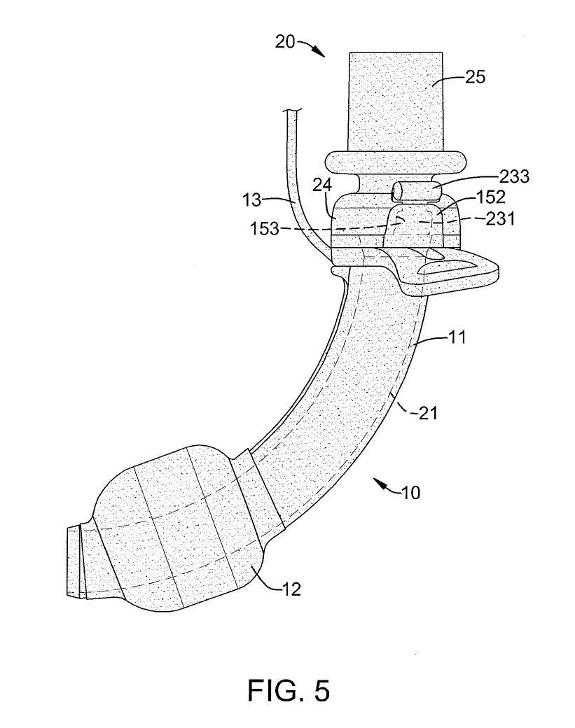

With reference to FIGS. 1 and 5, the airway tube 11 of the outer tube 10 is inserted into a patient's trachea through a stoma defined in the trachea by a surgical operation. A cord or strap can be mounted around the patient's neck to hold the neck plates 141 in place and the inflatable cuff 12 is inflated through the inflating tube 13 and abuts against an inner surface of the patient's trachea to hold the outer tube 10 securely in the neck of the patient. The connector 25 of the inner cannula 20 is connected to a ventilator or a respiratory tube, so that air can be forced directly into the trachea of the patient through the outer tube 10 and the inner cannula 20 of the inner type tracheostomy tube in accordance with the present invention.

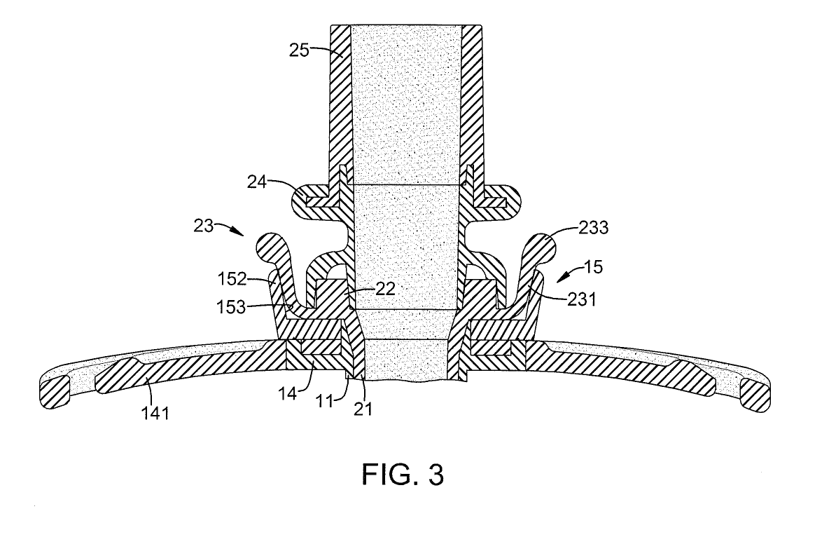

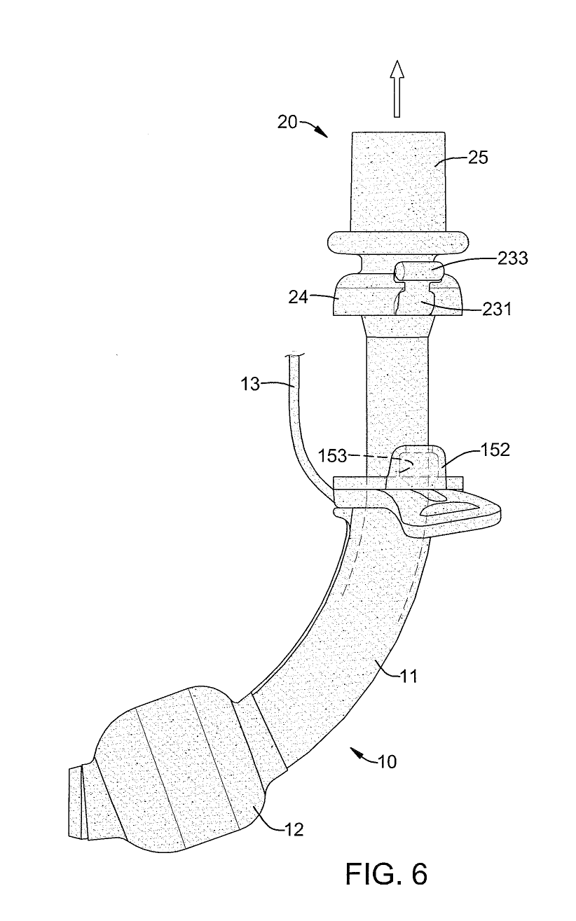

With reference to FIGS. 3 to 6, when the inner cannula 20 is needed to change, the pressing buttons 233 of the locking tabs 23 are pressed to separate the locking portions 231 of the locking tabs 23 respectively from the holding recesses 153 of the holding arms 152. Then, the inner tube 21 of the inner cannula 20 can be pulled out of the airway tube 11 of the outer tube 10 by pulling the pressing buttons 233 of the locking tabs 23 and a new inner cannula 20 can be inserted and held with the outer tube 10. Accordingly, to change an inner cannula 20 with a new one is convenient and easy.

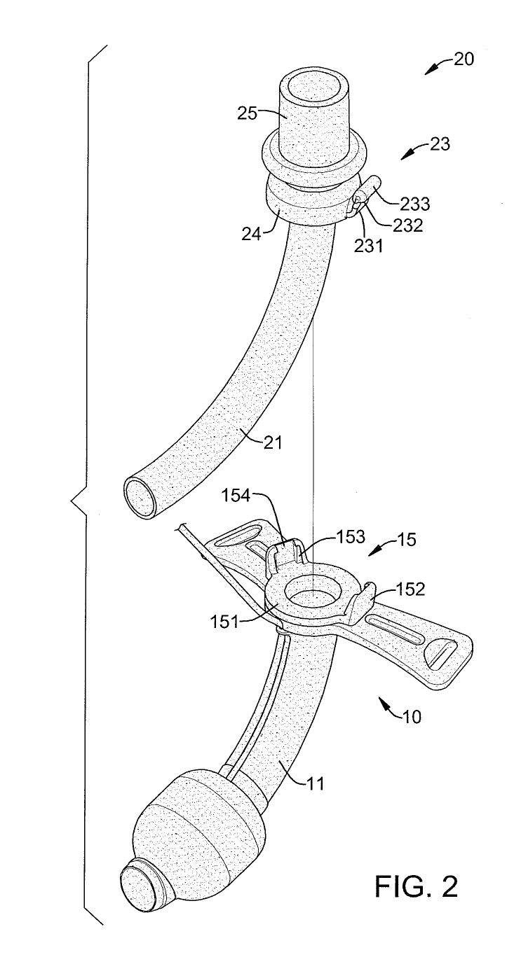

With further reference to FIG. 2, the locking portions 231 of the locking tabs 23 are respectively and securely mounted in the holding recesses 153 of the holding arms 152 and this can make the holding arms 152 mounting around the locking tabs 23 prevent dirt from accumulating on the locking tabs 23 and to avoid the patient knocking the locking tabs 23 of the inner cannula 20 to separate the inner cannula 20 from the outer tube 10 and this is clean and safe to use.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An inner type tracheostomy tube comprising
an outer tube having
an airway tube having
an inner end;
an outer end; and
an external surface;
an inflatable cuff mounted around the external surface near the inner end of the airway tube;
an inflating tube connected to and communicating with the inflatable cuff to inflate or deflate the inflatable cuff;
a neck mount securely mounted around the external surface of the airway tube near the outer end of the airway tube and having
a top; and
two sides; and
a holding mount formed on the neck mount around the outer end of the airway tube and having
a holding ring formed on the top of the neck mount around the outer end of the airway tube and having
an external surface; and
a top; and
two holding arms formed oppositely on the external surface of the holding ring and protruding from the top of the holding ring, and each holding arm having
a bottom formed on the external surface of the holding ring;
a top extending above the top of the holding ring;
an inner face; and
a holding recess formed in the inner face of the holding arm between the tops of the holding ring and the holding arm and having a shape and a width; and
an inner cannula detachably connected to the outer tube and having
an inner tube mounted in the airway tube of the outer tube and having
an inner end mounted in the airway tube near the inner end of the airway tube;
an outer end extending out of the holding ring of the holding mount; and
an external surface;
a disk segment formed on the external surface of the inner tube at the outer end of the inner tube, abutting the holding ring of the holding mount and having
a bottom abutting the top of the holding ring;
a top; and
an external surface;
two locking tabs formed on the external surface of the disk segment from the bottom of the disk segment and protruding from the top of the disk segment and respectively connected to the holding arms of the holding mount, and each locking tab having
a shape corresponding to that of the holding recess in a corresponding holding arm;
a lower end;
an upper end;
a middle;
a locking portion formed on the lower end of the locking tab, formed on the external surface of the disk segment and securely mounted in the holding recess of the corresponding holding arm and having a width; and
a pressing button formed on the upper end of the locking tab above the corresponding holding arm;
a connecting mount securely mounted around the disk segment between the locking tabs and having
a bottom end securely mounted around the disk segment between the locking tabs; and
a top end; and
a connector securely mounted around the top end of the connecting mount.

2. The inner type tracheostomy tube as claimed in claim 1, wherein
each holding recess is rectangular;
each holding arm has an opening formed through the top of the holding arm, communicating with the holding recess of the holding arm and having a width smaller than the width of the holding recess of the holding arm;
each locking tab has a neck portion formed on the middle of the locking tab, is connected with the locking portion of the locking tab and extending out of the opening of the corresponding holding arm and having a width smaller than the width of the locking portion of the locking tab; and
each pressing button is connected with a corresponding neck portion above the opening of a corresponding holding arm.

3. The inner type tracheostomy tube as claimed in claim 2, wherein
the neck mount is an annular hard board and has two neck plates being elongated and respectively formed on and protruding from the sides of the neck mount; and
the bottoms of the holding arms respectively abut the neck plates of the neck mount.

4. The inner type tracheostomy tube as claimed in claim 1, wherein
the neck mount is an annular hard board and has two neck plates being elongated and respectively formed on and protruding from the sides of the neck mount; and
the bottoms of the holding arms respectively abut the neck plates of the neck mount.
